(19) United States
(12) Patent Application Publication
(10) Pub. No.: US 2007/0225354 A1
(43) Pub. Date: Sep. 27, 2007

(54) Use of dihydrolipoamide for enhancing physical performance

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave, Detroit, MI, 48226, US

(21) Appl. No.: 11/385,074

(22) Filed: Mar. 21, 2006

(51) Int. Cl. A61K 31/385

(52) U.S. Cl. 514/440

(57) ABSTRACT

A method of decreasing fat and adipose tissue, reducing appetite and increasing alertness in humans comprises administering dihydrolipoamide in a daily dosage between about 300 and 2000 mg. Dihydrolipoamide may be administered once per day or in multiple doses, either alone or in combination with other compounds and mixtures.

### RELATED APPLICATION

This application claims priority benefit of U.S. provisional patent application No. 60/663,559 filed on Mar. 21, 2005.

### FIELD OF THE INVENTION

This invention relates to use of dihydrolipoamide as a supplement for enhancing physical performance.

### BACKGROUND OF THE INVENTION

Antioxidants play an important role in many body processes. Oxidation produces damaging free radicals (unpaired electrons or molecules having at least one unpaired electron). Inflammatory cells (granulocytes, macrophages, some T-lymphocytes, etc.) produce an active species of oxygen (one with an unpaired electron) as part of the microbiocidal/citocidal system. Excessive inflammation can harm surrounding tissues. Biological systems protect themselves against the damaging effects of activated species by several techniques. These include free radical scavengers and chain reaction terminators: enzymes such as SOD, catalase, and the glutathione peroxidase system. These techniques can be supplemented by the use of antioxidants. Antioxidants act by donating an electron to a free radical and converting it to a nonradical form. Such reducing compounds can terminate radical chain reactions and reduce hydroperoxides and epoxides to less reactive derivatives.

There are many antioxidants including Vitamins E and C, selenium, the carotenoids, etc. Vitamins E (alpha-tocopherol) and C (ascorbic acid) are also two vitamins necessary for proper bodily function they are not very strong antioxidants and are not broad-spectrum antioxidants, which means that they only work as antioxidants in some tissues of the body. Alpha-lipoic acid is another antioxidant which also acts as an insulin mimetic. Administration of alpha-lipoic acid can result in decreased blood glucose levels, reduced appetite, reduced adiposity and body weight. However, Alpha lipoic acid is unstable and degrades when exposed to light and air.

Obesity is a growing concern, particularly in the United States. Excessive body weight can lead to a variety of well known health problems, especially hypertension, heart disease, atherosclerosis, diabetes and strokes. Weight loss, specifically fat loss, is critical in maintaining a healthy body weight. Unfortunately, the sedate modern lifestyle leaves people consuming more calories than they need.

In addition to diet control and exercise, body weight may be decreased through the use of pharmaceuticals and dietary supplements. Sympathomimetics, (stimulators of the sympathetic nerves) like ephedrine and amphetamines have been used extensively for this purpose. Although such compounds have been found effective, they are associated with side effects including increased heart rate and blood pressure. It would be desirable to provide a relatively long lasting, robust, safe and effective new method for reducing weight, especially fat and adipose tissue, increasing endurance and increasing alertness.

### SUMMARY OF THE INVENTION

In accordance with a first aspect, method of decreasing fat and adipose tissue, decreasing appetite and increasing endurance in humans comprises administering dihydrolipoamide in a daily dosage between about 300 and 2000 mg.

From the foregoing disclosure and the following more detailed description of various preferred embodiments it will be apparent to those skilled in the art that the present invention provides a significant advance in the methods for reducing fat and adipose tissue, reducing appetite and increasing endurance. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the methods of use of dihydrolipoamide disclosed here. The following detailed discussion of various alternative and preferred features and embodiments will illustrate the general principles of the invention with reference to improved methods of enhancing physical performance through the use of orally available dietary supplements. Other embodiments suitable for other applications will be apparent to those skilled in the art given the benefit of this disclosure.

The present invention provides a method of administering to mammals, especially humans, dihydrolipoamide or one of its salts, esters or ethers for maintaining or increasing endurance, reducing appetite and/or decreasing fat mass.

Dihydrolipoamide is a naturally occurring compound which is structurally similar to alpha lipoic acid. The chemical structure of dihydrolipoamide is reproduced below.

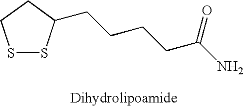

Dihydrolipoamide is the reduced form of lipoamide. Thus it can undergo oxidation, resulting in formation of lipoamide with an S—S bond replacing two —SH bonds. In addition, the amide group can be oxidized to form dihydrolipoic acid and upon further oxidation, lipoic acid. Therefore, dihydrolipoamide, being more reduced than any of these three compounds, has the greatest antioxidant potential. This advantageously increases dihydrolipoamide resistance to deactivation upon exposure to oxygen and ambient light during storage.

The method of using dihydrolipoamide to decrease body weight, especially reducing fat and adipose tissue, reducing appetite, and increasing endurance and alertness is a significant improvement over the use of sympathomimetics, ephedrine, amphetamines and the like in that it can achieve the positive effects without the addictive side effects.

Administration of dihydrolipoamide to an individual provides numerous benefits, not just as an anti-obesity agent, but also as a stable anti-oxidant, and insulin mimetic. Dihydrolipoamide can be administered one of the several ways: orally, transdermally, intranasally, by injection, sublingually, and transrectally as a suppository. Oral administration can comprise, for example, a liquid suspension, gel capsule or tablets. Tablets can be prepared by combining dihydrolipoamide with conventionally used binders and excipients, e.g. cellulose or cornstarch. Preferably a dosage would be about 300-2000 mg per day, most preferably about 500-1000 mg per day. Unit dosage can be about 250-500 mg or 125-250 mg of the compound, for example. Oral dosage forms could be administered in smaller divided doses multiple times per day (such as, two, three or four times per day) to help maintain more constant levels of dihydrolipoamide in the body.

Other compounds or mixtures may be added in combination with the dihydrolipoamide. For example caffeine or mixtures containing caffeine may be added, as caffeine help suppress appetite and aids in thermogenesis.

### EXAMPLE 1

Tablets. 1 kg of the compound dihydrolipoamide was mixed with microcrystalline cellulose, and placed into 2000 hard-gelatin capsules each with a content of 500 mg of dihydrolipoamide.

### EXAMPLE 2

Tablets. 5 kg dihydrolipoamide was mixed with 2.5 kg of green tea and placed into 10,000 hard-gelatin capsules each with a content of 500 mg of dihydrolipoamide and 250 mg of green tea.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of decreasing fat and adipose tissue in humans comprising administering dihydrolipoamide or one of its salts, esters or ethers in a daily dosage between about 300 mg and 2000 mg.

2. The method of claim 1 wherein dihydrolipoamide is administered in one of the following ways: orally, transdermally, intranasally, by injection, sublingually, and transrectally.

3. The method of claim 1 wherein dihydrolipoamide is administered in a daily dosage between about 500 mg and 1000 mg.

4. The method of claim 1 wherein dihydrolipoamide is administered in a dosage between about 250 mg and 500 mg two times per day.

5. The method of claim 1 wherein dihydrolipoamide is administered in a dosage between about 125 mg and 250 mg four times per day.

6. The method of claim 1 wherein dihydrolipoamide is administered in the form of one of a gel capsule, a tablet and a liquid suspension.

7. The method of claim 1 wherein dihydrolipoamide is administered in combination with caffeine.

8. The method of claim 1 wherein dihydrolipoamide is administered with green tea.

9. A method of reducing appetite in humans comprising administering dihydrolipoamide or one of its salts, esters or ethers in a daily dosage between about 300 mg and 2000 mg.

10. The method of claim 9 wherein dihydrolipoamide is administered with green tea.

11. A method of increasing endurance in humans comprising administering dihydrolipoamide or one of its salts, esters or ethers in a daily dosage between about 300 mg and 2000 mg.

12. The method of claim 11 wherein dihydrolipoamide is administered with green tea.
